(19) United States
(12) Patent Application Publication
(10) Pub. No.: US 2010/0198238 A1
(43) Pub. Date: Aug. 5, 2010

(54) APPARATUS AND METHODS FOR PERIVALVULAR LEAK OCCLUSION

(75) Inventor: Paul Sorajja, Rochester, MN (US)

Correspondence Address: MUETING, RAASCH & GEBHARDT, P.A., P.O. BOX 581336, MINNEAPOLIS, MN, 55458-1336, US

(73) Assignee: Mayo Foundation for Medical Education and Research, Rochester, MN (US)

(21) Appl. No.: 12/305,728

(22) PCT Filed: Jun. 19, 2007
(86) PCT No.: PCT/US07/14250

§ 371 Date: Sep. 28, 2009

Related U.S. Application Data
(60) Provisional application No. 60/814,820, filed on Jun. 19, 2006.

(51) Int. Cl. A61B 17/12

(52) U.S. Cl. 606/158

(57) ABSTRACT

Apparatus and methods for occluding perivalvular leaks located around the periphery of implanted replacement valves. The apparatus and methods may include both distal and proximal covers adapted for placement over a perivalvular leak, with the covers retained in position by tension between the proximal and distal flanges. The distal and proximal covers may be capable of collapsing into a delivery configuration amenable for delivery to an internal body location through a lumen of a delivery catheter and a deployment configuration in which a flange of the cover extends radially outward such that the flange defines a first major surface facing the opposing cover.

### RELATED APPLICATION

The present application claims the benefit of U.S. Provisional Patent Application Ser. No. 60/814,820, filed on Jun. 19, 2006 and titled APPARATUS AND METHODS FOR PERIVALVULAR LEAK OCCLUSION, which is hereby incorporated by reference in its entirety.

The present invention relates generally to implantable medical devices and, more particularly, to the occlusion of perivalvular leaks associated with, e.g., implanted replacement cardiac valves.

Cardiac valve replacement is well known in the art. The implanted valves may include, e.g., bioprosthetic or mechanical cardiac valves located in the aortic, mitral, pulmonary, or tricuspid positions. Although the valves may address serious deficiencies in cardiac function, the replacement valves may, as implanted, still suffer from leaks located about the periphery of the implanted valve. A leak or leaks located about the periphery of the implanted valve typically result in perivalvular regurgitation during use.

One approach to addressing perivalvular leaks is described in WO 2006/005015 (Spenser et al.). The devices and methods disclosed may suffer from a one or more disadvantages such as, e.g., requiring inflation, requiring one or more tissue anchors that may hinder removability of the device, etc. Furthermore, the devices are designed to be located within the cavity formed between the perimeter of the valve and the surrounding tissue. As such, the devices may potentially be subject to unwanted dislodgement after deployment.

### SUMMARY OF THE INVENTION

The present invention provides apparatus and methods for occluding perivalvular leaks located around the periphery of implanted replacement valves. The apparatus and methods may preferably include both distal and proximal covers adapted for placement over a perivalvular leak, with the covers retained in position by tension between the proximal and distal flanges. The distal and proximal covers are preferably capable of collapsing into a delivery configuration amenable for delivery to an internal body location through a lumen of a delivery catheter and a deployment configuration in which a flange of the cover extends radially outward such that the flange defines a first major surface facing the opposing cover.

The apparatus and methods may be used in, e.g., the repair of bioprosthetic or mechanical cardiac valves located in the aortic, mitral, pulmonary, or tricuspid positions. Although described herein for use in cardiac repair, the apparatus and methods may be used in the repair of other leaks and defects in other internal body locations.

In various embodiments, the flanges of the proximal and/or distal covers may preferably have noncircular perimeters to facilitate occlusion of a perivalvular defect while reducing simultaneous interference with or occlusion of the implanted replacement valve with which the apparatus is used.

In various embodiments, the proximal and/or distal covers may be rotatable about a longitudinal axis extending through the perivalvular leak. Rotation of the proximal and/or distal covers may be more beneficial if coupled with noncircular flanges to facilitate occlusion of a perivalvular defect while reducing simultaneous interference with (or occlusion of) the implanted replacement valve with which the apparatus is used.

In various embodiments, the apparatus of the present invention may be fully retrievable. Retrieval of the apparatus may be useful to, e.g., ascertain the efficacy of the apparatus at occluding the leak before finally deploying the apparatus within a patient.

It may be preferred that the apparatus of the present invention be compatible with conventional guide catheters, delivery catheters and imaging technology to facilitate deployment and proper positioning of the apparatus.

In one aspect, the present invention provides a perivalvular leak occlusion apparatus including a proximal cover; a distal cover; and a retention cable connecting the proximal cover to the distal cover, wherein the retention cable is in tension between the proximal cover and the distal cover and wherein the retention cable extends along a longitudinal axis of the apparatus between the proximal cover and the distal cover when under tension. The distal cover includes a distal hub to which the retention cable is attached and an expandable distal flange attached to the distal hub, wherein the distal flange is capable of collapsing into a delivery configuration amenable for delivery to an internal body location through a lumen of a delivery catheter, and wherein the distal flange is capable of moving from the delivery configuration into a deployment configuration in which the distal flange extends radially outward from the longitudinal axis and the distal hub such that the distal flange defines a first major surface facing the proximal cover. The proximal cover includes a proximal hub to which the retention cable is attached and an expandable proximal flange attached to the proximal hub, wherein the proximal flange is capable of collapsing into a delivery configuration amenable for delivery to an internal body location through a lumen of a delivery catheter, and wherein the proximal flange is capable of moving from the delivery configuration into a deployment configuration in which the proximal flange extends radially outward from the longitudinal axis and the proximal hub such that the proximal flange defines a first major surface facing the distal cover.

In various embodiments, the perivalvular leak occlusion apparatus of the invention may include one or more of the following features: the distal flange may include a structural framework and a membrane attached to the structural framework; the proximal flange may include a structural framework and a membrane attached to the structural framework; the membrane of the distal and/or proximal flange may include a polymeric film; the structural framework of the of the distal flange and/or proximal flange may include shape memory material; the first major surface of the distal flange may have a non-circular perimeter when the distal flange is in the deployment configuration; the first major surface of the proximal flange may have a non-circular perimeter when the proximal flange is in the deployment configuration, etc.

In some embodiments, the perivalvular leak occlusion apparatus may have a distal hub that includes a first element, a second element and a cinching element, wherein the cinching element has an orifice through which the retention cable extends, and further wherein the cinching element is adapted for non-reversible movement in the distal direction over the retention cable, and further wherein the cinching element is adapted to retain the distal flange in the deployment configuration. The first element and the second element may be spaced apart from each other along the retention cable when the distal flange is in the delivery configuration, wherein the second element is fixed at a selected location along the retention cable, and further wherein the first element moves along the retention cable towards the second element as the distal flange moves from the delivery configuration to the deployment configuration.

In some embodiments, the perivalvular leak occlusion apparatus may have a proximal hub that may include a first element, a second element and a cinching element, wherein the cinching element has an orifice through which the retention cable extends, and further wherein the cinching element is adapted for non-reversible movement in the distal direction over the retention cable, and further wherein the cinching element is adapted to retain the proximal flange in the deployment configuration. The first element and the second element may be spaced apart from each other along the retention cable when the proximal flange is in the delivery configuration, wherein the first element and the second element are closer to each other when the proximal flange is in the deployment configuration. The first element and the second element may have complementary shapes such that rotation of the first element causes corresponding rotation of the second element, wherein the proximal flange can be rotated about the retention cable. The cinching element of the proximal hub may maintain the retention cable in tension when the apparatus is in the deployed configuration.

In another aspect, the present invention provides a method of occluding a perivalvular defect by providing perivalvular leak occlusion apparatus of the present invention; advancing the distal cover of the apparatus to a distal side of the perivalvular defect; deploying the distal cover over the distal side of the perivalvular defect; advancing the proximal cover of the apparatus to the proximal side of the perivalvular defect; deploying the proximal cover over the proximal side of the perivalvular defect; and retaining the distal cover and the proximal cover in place over the distal and proximal sides of the perivalvular defect using the retention cable; wherein the retention cable is under tension between the distal cover and the proximal cover.

The methods of the present invention may, in various embodiments include one or more of the following: the first major surface of the distal flange may have a non-circular perimeter when the distal cover is deployed, wherein the method may include rotating the distal cover about the longitudinal axis to a selected orientation in which the distal cover does not interfere with operation of an implanted replacement valve; the first major surface of the proximal flange may have a non-circular perimeter when the proximal cover is deployed, wherein the method may include rotating the proximal cover about the longitudinal axis to a selected orientation in which the proximal cover does not interfere with operation of an implanted replacement valve; releasing the tension on the retention cable to remove the apparatus from the perivalvular defect; placing the proximal cover into its delivery configuration for removal from the perivalvular defect using a catheter; placing the distal cover into its delivery configuration for removal from the perivalvular defect using a catheter; using a support wire having an end loop extending past the distal cover; removing the support wire from the apparatus after deployment of the apparatus; etc.

The above summary is not intended to describe each embodiment or every implementation of the present invention. Rather, a more complete understanding of the invention will become apparent and appreciated by reference to the following Detailed Description of Exemplary Embodiments and claims in view of the accompanying figures of the drawing.

### BRIEF DESCRIPTIONS OF THE VIEWS OF THE FIGURE

FIGS. 1A-1E depict one exemplary deployment method and device for addressing a perivalvular defect in accordance with the present invention.

FIGS. 2A-2D depict exemplary delivery and deployment of exemplary proximal covers that may be used in connection with the present invention.

FIGS. 3A-3C depict an exemplary cinching element that may be used in connection with the present invention.

FIGS. 4A-4D depict an exemplary embodiment of a distal cover and its deployment.

FIGS. 5A-5D depict another exemplary embodiment of a proximal cover and its deployment in connection with the present invention.

FIG. 6 depicts some potentially suitable noncircular perimeter shapes for the proximal and/or distal covers that may be used in connection with the present invention.

FIGS. 7A-7C depict one exemplary method in which covers with non-circular perimeters may be rotated during deployment.

FIGS. 8A & 8B depict one exemplary structure that may be used to rotate covers in apparatus and/or methods of the present invention.

FIG. 9 depict an apparatus that includes an optional support wire to assist in deployment/retention of the apparatus of the present invention.

### DESCRIPTION OF EXEMPLARY EMBODIMENTS OF THE INVENTION

In the following description of exemplary embodiments of the invention, reference is made to the accompanying figures of the drawing which form a part hereof, and in which are shown, by way of illustration, specific exemplary embodiments in which the invention may be practiced. It is to be understood that other embodiments may be utilized and structural changes may be made without departing from the scope of the present invention.

As discussed herein, the present invention provides apparatus and methods useful in treating perivalvular leaks located around the periphery of implanted replacement valves. The location of the defect(s) may, e.g., be identified by echocardiography (intracardiac, transesophageal, transthoracic, or combination thereof) and/or invasive angiography. Both of these techniques may then be utilized adjunctively to confirm positioning of the apparatus of the invention before, during, and after deployment.

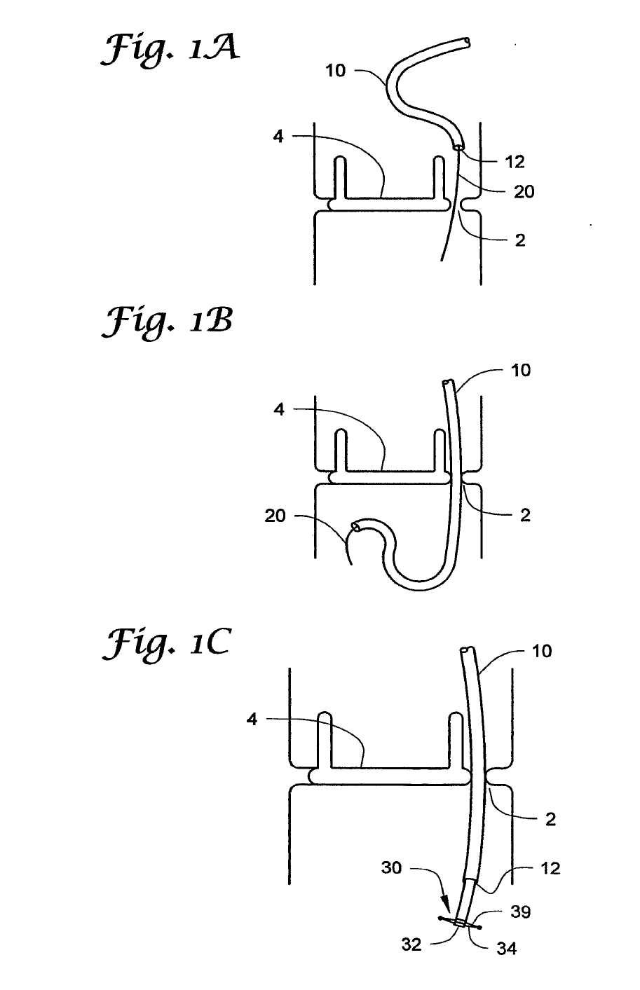

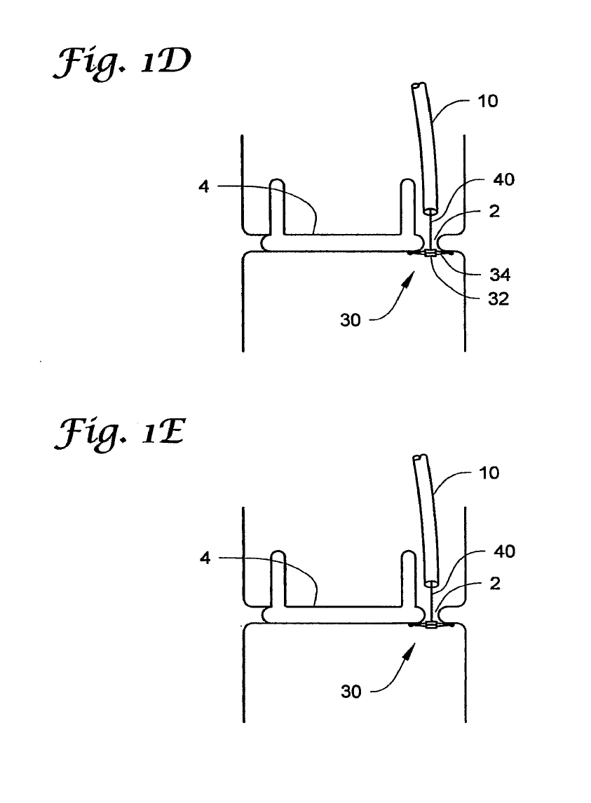

One deployment method is depicted in FIGS. 1A-1E in which a perivalvular defect (leak) 2 is located about the periphery of an implanted replacement valve 4. Following identification of a perivalvular leak 2, a guide catheter 10 (e.g., a standard 6 to 10 French cardiac guide catheter—the use of either a preformed Amplatz, multi-purpose, or right Judkins catheter may normally suffice, but the appropriate catheter utilized will depend on the location of the defect) may be used to either directly engage the perivalvular leak 2 or be positioned in the proximity of the leak 2 to facilitate advancement of a guidewire through the leak 2.

With the guide catheter 10 positioned with its distal end 12 near the leak 2, a guidewire 20 (e.g., an exchange-length straight wire having a diameter of, e.g., 0.025″ to 0.038″) may be advanced through the leak 2 as depicted in FIG. 1A. After the guidewire 20 crosses the leak 2, the guide catheter 10 may preferably be advanced across the leak 2 as depicted in FIG. 1B. With the guide catheter 10 in position across the leak 2, the guidewire 20 may preferably be removed from the guide catheter 10, leaving the distal end 12 of the guide catheter 10 located distal to the leak 2. After each crossing of the leak 2 with either the guidewire 20 or the guide catheter 10, adjunctive echocardiography may preferably be employed to confirm the correct position of the devices across the leak 2.

With the guide catheter 10 in position across the leak 2, deployment of the components of the apparatus of the present invention may begin. As depicted in FIG. 1C, a distal cover 30 of the apparatus of the present invention may preferably be advanced through the guide catheter 10 until it exits from the distal end 12 of the guide catheter 10. The distal cover 30 is depicted in its deployed configuration in FIG. 1C, where the distal cover 30 is expanded.

The distal cover 30 may preferably include a hub 32 from which a flange 34 preferably extends radially outward from a longitudinal axis defined by, e.g., the retention cable 40 such that the flange 34 defines a major surface 39 facing the distal side of the leak 2.

After expansion of the distal cover 30 outside of the guide catheter 10, the distal cover 30 may preferably be drawn back in the proximal direction until it is seated on the distal side of the leak 2 as depicted in FIG. 1D. It may be preferable to employ echocardiography and/or invasive angiography to confirm absence of regurgitation while the leak 2 is covered by the distal cover. Adjunctive echocardiographic imaging may also preferably be used to ensure that the implanted replacement valve is not unacceptably obstructed with the distal cover 30 in place over the leak 2. Following satisfactory evaluation of the implanted replacement valve 4 and the leak 2 with the distal cover 30 in place, the guide catheter 10 may be retracted over a retention cable 40 that remains attached to the distal cover 30 as depicted in FIG. 1E.

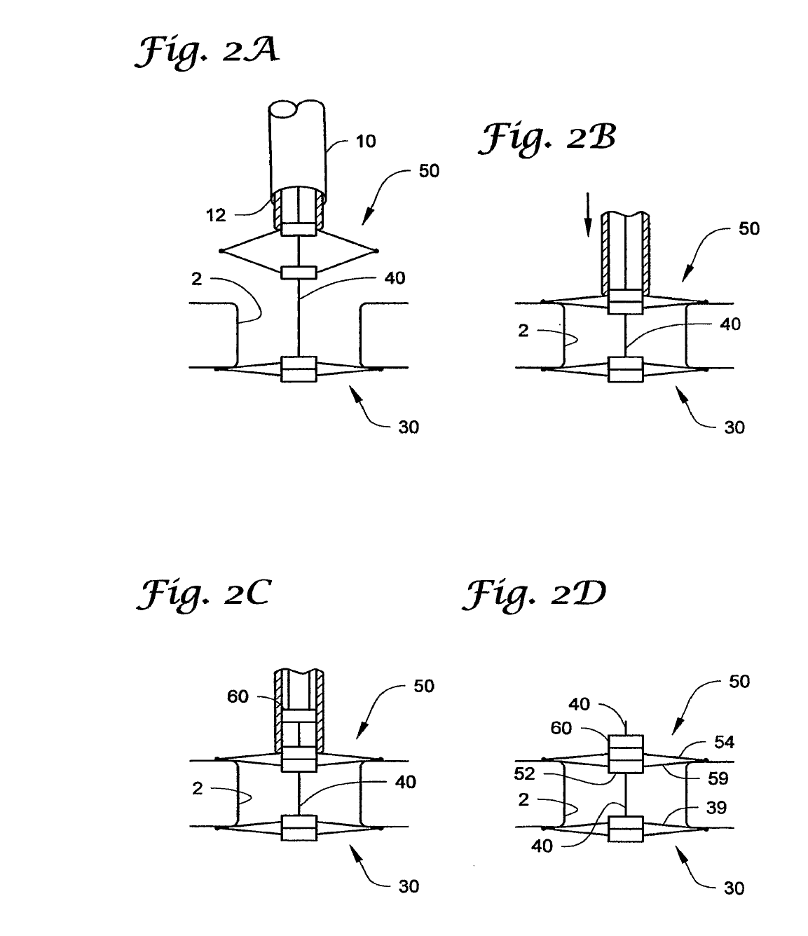

Delivery and deployment of a proximal cover 50 is depicted in FIGS. 2A-2D. With the guide catheter 10 still in position near the proximal side of the leak 2 as seen in FIG. 1E, a proximal cover 50 may preferably be advanced through the guide catheter 10 and over the retention cable 40 until it exits the distal end 12 of the guide catheter 10 as depicted in FIG. 2A.

After (or while) the proximal cover 50 exits the distal end 12 of the guide catheter 10, the proximal cover 50 preferably expands from its delivery configuration (in which it travels through the guide catheter 10) to its deployment configuration. In FIG. 2A, for example, the proximal cover 50 is only partially expanded to its deployment configuration.

After the proximal cover 50 is deployed outside of the guide catheter 10, it is preferably moved into place along the retention cable 40 until seated at the proximal side of the leak 2 as depicted in FIG. 2B. Echocardiography and/or invasive angiography may again be used to confirm absence of regurgitation while the leak 2 and/or to ensure that the implanted replacement valve is not unacceptably obstructed with the proximal cover 50 in place over the proximal side of the leak 2.

Following satisfactory evaluation of the implanted replacement valve and the leak 2 with both the proximal cover 50 and the distal cover 30 in place, a cinching element 60 may preferably be advanced through the guide catheter 10 over the retention cable 40 to a position on the proximal side of the proximal cover 50 as, e.g., depicted in FIG. 2C. The cinching element 60 may preferably be capable of holding the retention cable 40 in tension between the distal cover 30 and the proximal cover 50. The tension provided by the retention cable 40 in conjunction with the cinching element 60 is preferably sufficient to hold the distal cover 30 and the proximal cover 50 in place over the distal and proximal sides of the leak 2 as depicted in FIG. 2D.

With the distal cover 30 and the proximal cover 50 in place over the leak 2 and the proper amount of tension on the retention cable 40, it may be preferred, as depicted in FIG. 2D, to sever the retention cable 40 proximally of the cinching element 60, leaving the apparatus (the distal cover 30, proximal cover 50 and retention cable 40 extending therebetween) in place. The guide catheter 10 and remainder of the retention cable 40 may then preferably be removed, leaving the occlusion apparatus in place in leak 2.

As deployed, the proximal cover 50 may preferably include a hub 52 from which a flange 54 preferably extends radially outward from a longitudinal axis defined by, e.g., the retention cable 40 such that the flange 54 defines a major surface 59 facing the proximal side of the leak 2 as well as the major surface 39 of the distal cover 30 in position on the distal side of the leak 2.

In some embodiments, the deployment process may be reversible. In other words, it may preferably be possible to release the tension on the retention cable 40 and remove the distal cover 30 and the proximal cover 50. Removal of the distal cover 30 and the proximal cover 50 may preferably involve moving the covers back into their respective delivery configurations such that they can be drawn into a catheter for removal from the subject.

The cinching element 60 may take a variety of forms with only one form being depicted in FIGS. 2C & 2D. It may be preferred that the cinching element 60 perform the primary functions of retaining the proximal cover 50 against the proximal side of the leak while also retaining the retention cable 40 in tension. Any structure or structures capable of performing those two functions may be used.

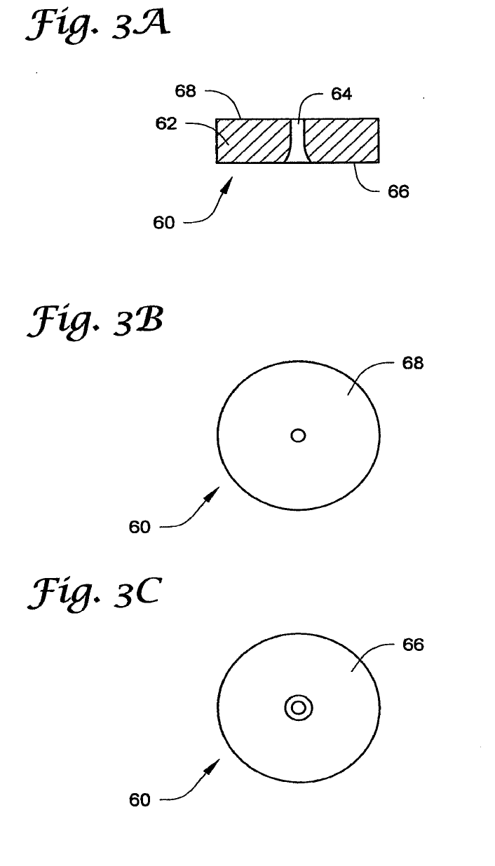

One example of a suitable structure for a cinching element 60 is depicted in FIGS. 3A-3C. The depicted cinching element 60 includes a body 62 having an orifice 64 through which the retention cable 40 passes. The orifice 64 may preferably be constructed such that the cinching element 60 moves along the retention cable 40 in one direction with significantly less resistance than in the opposite direction. In the depicted embodiment, the orifice 64 is larger on the distal side 66 and decreases in size toward the proximal side 68 of the body 62. The smaller opening of the orifice 64 is preferably capable of exerting sufficient friction on the retention cable 40 to resist movement of the cinching element in the proximal direction along a cable. The orifice 64 may, in some instances, include coatings, ridges, etc. that facilitate the ability of the cinching element 60 to resist movement in the proximal direction.

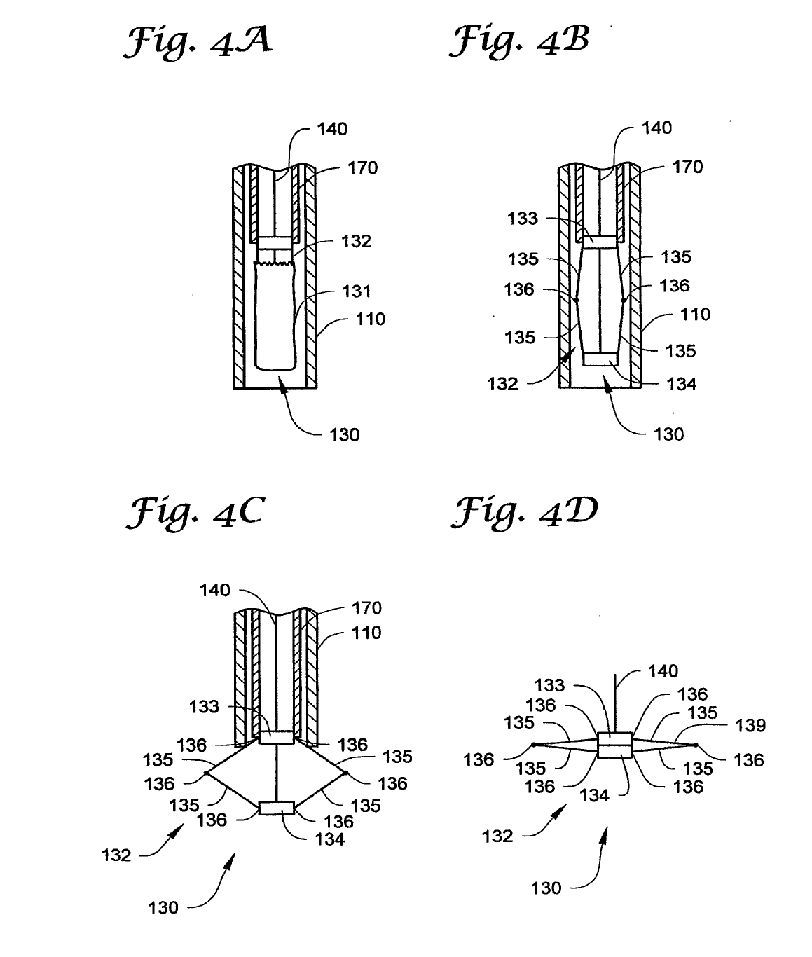

Although the distal covers used in apparatus according to the present invention may take a variety of forms, one exemplary embodiment of a distal cover is depicted in FIGS. 4A-4D. The distal cover may preferably be in the form of a structural framework with a membrane attached to the structural framework. The structural framework may preferably support the membrane across a perivalvular defect to reduce or prevent unwanted flow through the defect.

The distal cover may preferably include both a delivery configuration in which the distal cover is adapted for delivery to an internal body location through a lumen of a guide catheter. The distal cover is depicted in a delivery configuration in FIG. 4A, with the membrane 131 wrapped around the structural framework 132. The membrane 131 may preferably be folded or pleated such that the membrane 131 is small enough to fit within the guide catheter 110 as seen in FIG. 4A, yet can expand to a size large enough to cover the defect to be closed. One example of a potentially suitable folding pattern may be found in, e.g., a pleated drip coffee filter

Some of the membranes used in connection with the present invention may be constructed from synthetic or natural materials. Some potentially suitable natural materials may include, e.g., porcine pericardium, human pericardium, albumin, collagen, fibrin-based membranes, etc. Some potentially suitable synthetic membrane materials may include, e.g., cyanoacrylates, polytetrafluoroethylene, etc.

Still other membranes may be provided in the form of a porous or mesh body that may be designed to promote cell ingrowth after implantation. Some potentially suitable constructions may include, e.g., non-woven materials, woven materials, knitted materials, metallic (or other) matrices, etc. Porous membranes may be provided in combination with materials that promote cellular ingrowth, e.g., cell recruitment factors (VEGF, EGF, FGF, PDGF, etc.).

Other membranes used in connection with the present invention may be constructed of degradable materials such that, over time, the amount of membrane material at the deployment site would be reduced (e.g., it may be replaced by tissue). For example, the membrane could be constructed of a degradable bio-polymer.

FIG. 4B depicts the distal cover 130 in the delivery configuration within the guide catheter 110 with the membrane removed to allow for visualization of the structural framework 132. The proximal end 133 of the distal cover 130 may preferably be attached to a delivery catheter 170 that is adapted to be advanced through the guide catheter 110, although other delivery apparatus may be used in place of the delivery catheter 170.

The structural framework 132 used to support and/or expand the membrane 131 may preferably include a proximal end 133 and a distal end 134. The proximal end 133 and the distal end 134 may preferably be connected to each other by struts 135 that are arranged and connected to serve as a structural framework capable of supporting and retaining a membrane over a perivalvular defect as discussed herein.

It may be preferred that the struts 135 be connected by hinges 136. The hinges 136 may be provided as distinct structural devices (e.g., including a pin, etc.) connecting separate and distinct struts 135. Alternatively, if the structural framework 132 is provided from e.g., a shape memory material, the hinges 136 may be formed by integral folds or bends in the struts 135 that take the desired shape as the structural framework 132 of the distal cover expands into the deployment configuration from the delivery configuration as discussed herein.

The proximal end 133 and the distal end 134 may preferably both be connected to a retention cable 140 as depicted in FIGS. 4A-4D. It may he preferred that, for the depicted distal cover, the distal end 134 be fixedly attached at a selected location along the retention cable 140 while the proximal end 133 be mounted over the retention cable 140 such that it can move along the length of the retention cable 140 (sometimes referred to herein as the longitudinal axis defined by the retention cable 140).

As the structural framework 132 of the distal cover advances out of the confines of the guide catheter 110, the struts 135 begin to expand and the distance between proximal end 133 and the distal end 134 of the cover decreases from the distance with which they are separated in the delivery configuration as depicted in FIG. 4C. As the proximal end 133 and the distal end 134 approach each other, the flattened struts 135 (and membrane—not shown) preferably form a flange about the hub formed by the combination of the proximal end 133 and distal end 134. The proximal end 133 and the distal end 134 may preferably be in contact with each other when the structural framework 132 is in the fully deployed configuration as depicted in FIG. 4D. When in the deployed configuration, the distal cover preferably defines a first major surface 139 that faces the proximal cover (not shown) of an apparatus in which a proximal cover is used.

The distal cover may preferably be retained in the deployment configuration of FIG. 4D by the struts 135. Alternatively, a cinching element may be advanced along the retention cable 140 to retain the distal cover in the deployed configuration.

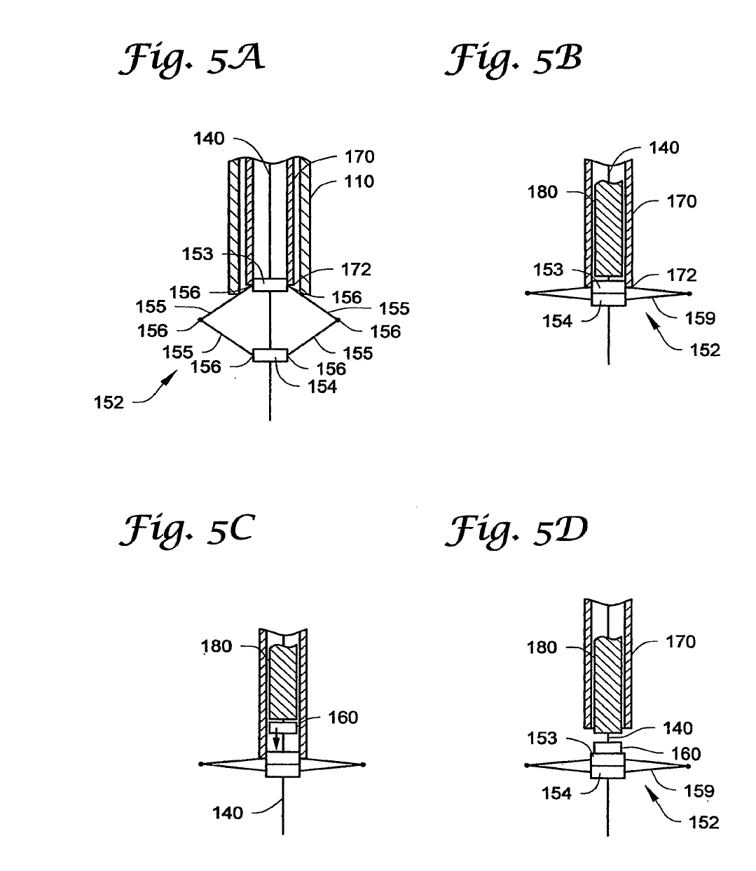

One exemplary embodiment of a proximal cover that may be used in connection with the apparatus and methods of the present invention is depicted in FIGS. 5A-5D. In many respects, the proximal cover may have a construction similar to the distal cover depicted in connection with FIGS. 4A-4D. The proximal cover of FIG. 5A may, e.g., preferably be in the form of a structural framework with a membrane attached to the structural framework. The structural framework may preferably support the membrane across the proximal side of a perivalvular defect to reduce or prevent unwanted flow through the defect.

Like the distal cover, the proximal cover may also preferably include both a delivery configuration in which the distal cover is adapted for delivery to an internal body location through a lumen of a guide catheter. The proximal cover is depicted in a partially deployed configuration in FIG. 5A as the cover leaves the confines of the guide catheter 110. Although not depicted, it should be understood that the proximal cover also preferably includes a membrane attached to the structural framework 152 (as described in connection with the distal cover of FIGS. 4A-4D).

The structural framework 152 used to support and/or expand the membrane may preferably include a proximal end 153 and a distal end 154. The proximal end 153 and the distal end 154 may preferably be connected to each other by struts 155 that are arranged and connected to serve as a structural framework capable of supporting and retaining a membrane over a perivalvular defect as discussed herein.

It may be preferred that the struts 155 be connected by hinges 156. The hinges 156 may be provided as distinct structural devices (e.g., including a pin, etc.) connecting separate and distinct struts 155. Alternatively, if the structural framework 152 is provided from e.g., a shape memory material, the hinges 156 may be formed by integral folds or bends in the struts 155 that take the desired shape as the proximal cover expands into the deployment configuration from the delivery configuration as discussed herein.

The proximal end 153 and the distal end 154 may preferably both be connected to a retention cable 140 as depicted in FIGS. 5A-5D. In one difference between the proximal cover of FIGS. 5A-5D and the distal cover of FIGS. 4A-4D, it may be preferred that, for the depicted proximal cover, both the proximal end 153 and the distal end 154 be mounted over the retention cable 140 such that both ends 153 and 154 can move along the length of the retention cable 140 (sometimes referred to herein as the longitudinal axis defined by the retention cable 140).

FIG. 5B depicts the proximal cover after the structural framework 152 is preferably fully expanded into the deployment configuration. The proximal end 153 is depicted as still attached within the distal end 172 of a delivery catheter 170 that is adapted to be advanced through the guide catheter 110, although other delivery apparatus may be used in place of the delivery catheter 170.

With the proximal end 153 and the distal end 154 proximate each other as seen in FIG. 5B, the flattened struts 155 (and membrane—not shown) preferably form a flange about the hub formed by the combination of the proximal end 153 and distal end 154. The proximal end 153 and the distal end 154 may preferably be in contact with each other when the structural framework 152 is in the deployed configuration as depicted in FIGS. 5B-5D. When in the deployed configuration, the proximal cover preferably defines a first major surface 159 that faces the distal cover (not shown) of an apparatus in which a proximal cover is used.

It may be preferred that a cinching element 160 be advanced along the retention cable 140 to prevent the proximal end 153 and the distal end 154 from moving in the proximal direction along the retention cable 140. The cinching element 160 may be advanced along the retention cable 140 by a pushing catheter 180 as depicted in FIG. 5D.

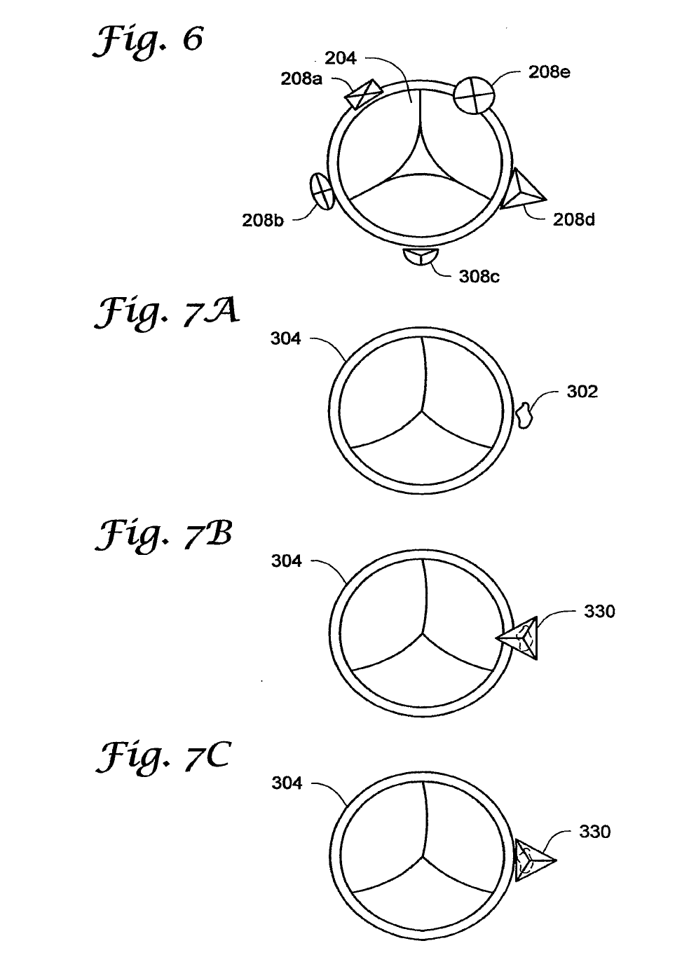

It may be preferred that the proximal and/or distal covers used in connection with the apparatus of the present invention have major surfaces (when in the deployed configuration) that have a non-circular perimeter. Examples of some potentially suitable non-circular perimeter shapes are depicted in FIG. 6 relative to an implanted replacement valve 204. The non-circular shapes may include, e.g., rectangle 208a, oval 208b, semicircle 208c, and triangle 208d. Although non-circular perimeters may be preferred, covers with circular perimeters may also be used—one example of which is depicted as cover 208e in FIG. 6.

Covers with non-circular perimeter shapes may be preferred over circular shapes because the non-circular perimeter shapes may be less likely to obstruct or interfere with operation of the valve 204. As depicted in FIG. 6, for example, the circular cover 208e overlaps a portion of the valve 204 and, as a result, may interfere with proper operation of the valve 204. In contrast, the covers with non-circular perimeter shapes may preferably be oriented such that the covers are less likely to overlap and interfere with operation of the valve 204.

If the covers used in apparatus of the present invention have non-circular perimeters, it may be desirable to be able to rotate the cover about the axis defined by the retention cable used to secure the apparatus in place over a defect. FIGS. 7A-7C depict one such method in which a perivalvular defect 302 is located proximate an implanted replacement valve 304 as seen in FIG. 7A. A cover 330 having a triangular perimeter may be positioned over the defect as depicted in FIG. 7B. In the orientation depicted in FIG. 7B, however, the cover 330 may be positioned over a portion of the valve 304. In such a situation, it may be desirable to rotate the cover 330 such that it no longer overlaps the valve as depicted in FIG. 7C.

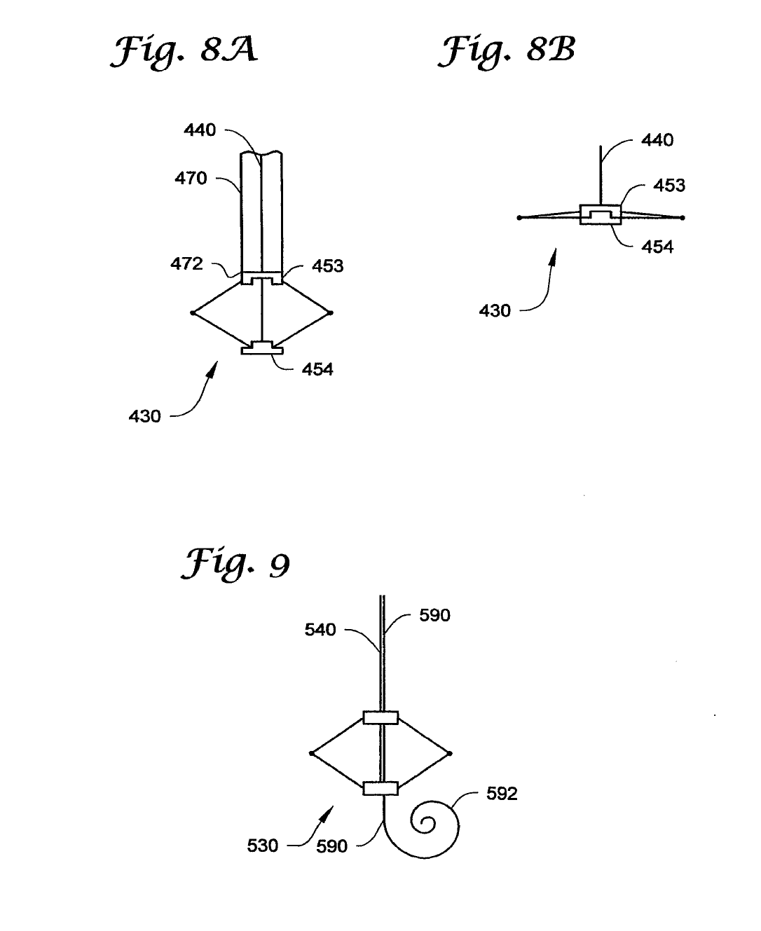

Rotation of the covers used in apparatus of the present invention may be accomplished by any suitable technique or structure. One exemplary structure that may be used to effect rotation of the covers of the present invention is depicted in FIGS. 8A & 8B. The cover 430 depicted in FIGS. 8A & 8B may preferably be located along retention cable 440 as discussed herein and include a structural framework 452 extending outward from a proximal end 453 and a distal end 454. The hub formed by the proximal end 453 and the distal end 454 may preferably interlock with, e.g., the distal end 472 of a delivery catheter 470 to facilitate rotation of the cover 430 about a longitudinal axis defined by the retention cable 440.

To facilitate stabilization during deployment of the proximal and/or distal covers used in the apparatus of the present invention, a support wire 590 that extends through the cover 530 (or covers) may be supplied in addition to the retention cable 540 as depicted in FIG. 9. The support wire 590 may preferably be substantially stiffer than, e.g., retention cable 540 and may preferably include an end loop 592 to, e.g., reduce ectopy in the left ventricle during device deployment. Such a support wire 590, if used, may preferably be removed following complete deployment of the apparatus. Any lumens or other openings made to accommodate the support wire may preferably be closed after implantation by e.g., tissue ingrowth (endothelialization, etc.).

The complete disclosure of the patents, patent documents, and publications cited herein are incorporated by reference in their entirety as if each were individually incorporated.

Exemplary embodiments of this invention are discussed and reference has been made to possible variations within the scope of this invention. These and other variations and modifications in the invention will be apparent to those skilled in the art without departing from the scope of the invention, and it should be understood that this invention is not limited to the exemplary embodiments set forth herein. Accordingly, the invention is to be limited only by the claims provided below and equivalents thereof.

1. A perivalvular leak occlusion apparatus comprising:
a proximal cover;
a distal cover; and
a retention cable connecting the proximal cover to the distal cover, wherein the retention cable is in tension between the proximal cover and the distal cover and wherein the retention cable extends along a longitudinal axis of the apparatus between the proximal cover and the distal cover when under tension;
wherein the distal cover comprises:
a distal hub to which the retention cable is attached;
an expandable distal flange attached to the distal hub, wherein the distal flange is capable of collapsing into a delivery configuration amenable for delivery to an internal body location through a lumen of a delivery catheter, and wherein the distal flange is capable of moving from the delivery configuration into a deployment configuration in which the distal flange extends radially outward from the longitudinal axis and the distal hub such that the distal flange defines a first major surface facing the proximal cover;
and wherein the proximal cover comprises:
a proximal hub to which the retention cable is attached;
an expandable proximal flange attached to the proximal hub, wherein the proximal flange is capable of collapsing into a delivery configuration amenable for delivery to an internal body location through a lumen of a delivery catheter, and wherein the proximal flange is capable of moving from the delivery configuration into a deployment configuration in which the proximal flange extends radially outward from the longitudinal axis and the proximal hub such that the proximal flange defines a first major surface facing the distal cover.

2. An apparatus according to claim 1, wherein the distal flange comprises a structural framework and a membrane attached to the structural framework.

3. An apparatus according to claim 1, wherein the proximal flange comprises a structural framework and a membrane attached to the structural framework.

4. An apparatus according to claim 2, wherein the membrane comprises a polymeric film.

5. An apparatus according to claim 2, wherein the structural framework comprises shape memory material.

6. An apparatus according to claim 1, wherein the first major surface of the distal flange comprises a non-circular perimeter when the distal flange is in the deployment configuration.

7. An apparatus according to claim 1, wherein the first major surface of the proximal flange comprises a non-circular perimeter when the proximal flange is in the deployment configuration.

8. An apparatus according to claim 1, wherein the distal hub comprises a first element, a second element and a cinching element, wherein the cinching element comprises an orifice through which the retention cable extends, and further wherein the cinching element is adapted for non-reversible movement in the distal direction over the retention cable, and further wherein the cinching element is adapted to retain the distal flange in the deployment configuration.

9. An apparatus according to claim 8, wherein the first element and the second element are spaced apart from each other along the retention cable when the distal flange is in the delivery configuration, and wherein the second element is fixed at a selected location along the retention cable, and further wherein the first element moves along the retention cable towards the second element as the distal flange moves from the delivery configuration to the deployment configuration.

10. An apparatus according to claim 1, wherein the proximal hub comprises a first element, a second element and a cinching element, wherein the cinching element comprises an orifice through which the retention cable extends, and further wherein the cinching element is adapted for non-reversible movement in the distal direction over the retention cable, and further wherein the cinching element is adapted to retain the proximal flange in the deployment configuration.

11. An apparatus according to claim 10, wherein the first element and the second element are spaced apart from each other along the retention cable when the proximal flange is in the delivery configuration, and wherein the first element and the second element are closer to each other when the proximal flange is in the deployment configuration.

12. An apparatus according to claim 10, wherein the first element and the second element comprise complementary shapes such that rotation of the first element causes corresponding rotation of the second element, wherein the proximal flange can be rotated about the retention cable.

13. An apparatus according to claim 10, wherein the cinching element of the proximal hub maintains the retention cable in tension when the apparatus is in the deployed configuration.

14. A method of occluding a perivalvular defect, the method comprising:
advancing the distal cover of an apparatus according to claim 1 to a distal side of the perivalvular defect;
deploying the distal cover over the distal side of the perivalvular defect;
advancing the proximal cover of the apparatus to the proximal side of the perivalvular defect;
deploying the proximal cover over the proximal side of the perivalvular defect; and
retaining the distal cover and the proximal cover in place over the distal and proximal sides of the perivalvular defect using the retention cable; wherein the retention cable is under tension between the distal cover and the proximal cover.

15. A method according to claim 14, wherein the first major surface of the distal flange comprises a non-circular perimeter when the distal cover is deployed, and wherein the method further comprises rotating the distal cover about the longitudinal axis to a selected orientation in which the distal cover does not interfere with operation of an implanted replacement valve.

16. A method according to claim 14, wherein the first major surface of the proximal flange comprises a non-circular perimeter when the proximal cover is deployed, and wherein the method further comprises rotating the proximal cover about the longitudinal axis to a selected orientation in which the proximal cover does not interfere with operation of an implanted replacement valve.

17. A method according to claim 14, further comprising releasing the tension on the retention cable to remove the apparatus from the perivalvular defect.

18. A method according to claim 17, further comprising placing the proximal cover into its delivery configuration for removal from the perivalvular defect using a catheter.

19. A method according to claim 17, further comprising placing the distal cover into its delivery configuration for removal from the perivalvular defect using a catheter.

20. A method according to claim 14, the method further comprising using a support wire comprising an end loop extending past the distal cover.

21. A method according to claim 20, further comprising removing the support wire from the apparatus after deployment of the apparatus.
